(19) United States
(12) Patent Application Publication
(10) Pub. No.: US 2026/0298728 A1
(43) Pub. Date: Oct. 1, 2026

(54) SYSTEM AND METHOD FOR AUTOMATICALLY CORRECTING VOLTAGE READINGS FROM A RADIOFREQUENCY PROBE CONNECTED TO A RADIOFREQUENCY GENERATOR

(71) Applicant: Boston Scientific Scimed, Inc., Maple Grove, MN (US)

(72) Inventors: Jin Yu, Valley Village, CA (US); Raul Enrique Serrano Carmona, Los Angeles, CA (US); Alexander Pruitt, San Jose, CA (US); Raman Taneja, Studio City, CA (US)

(21) Appl. No.: 19/572,666

(22) Filed: Mar. 19, 2026

Related U.S. Application Data
(60) Provisional application No. 63/778,048, filed on Mar. 26, 2025.

(51) Int. Cl. G01K 7/13; A61B 18/00; A61B 18/12; A61B 18/14

(52) U.S. Cl. CPC G01K 7/13; A61B 18/1206; A61B 18/14; A61B 2018/00577; A61B 2018/00767; A61B 2018/00821; G01K 2219/00

(57) ABSTRACT

Systems and methods for improving temperature measurement accuracy in radiofrequency ablation systems while eliminating manufacturing calibration procedures. The systems include a radiofrequency generator with an analog-to-digital converter (ADC) that converts thermocouple analog voltage signals into digital converter voltage signals. The system determines thermocouple type, applies a first correction to digital converter voltage signals, applies a second correction to the converted cold junction voltage signals to compensate for sensor location offset, determines an adjusted voltage based on these corrected signals, and converts to a temperature using a standard lookup table corresponding to the thermocouple type. This approach eliminates individual device calibration during manufacturing while accommodating both T-type and K-type thermocouples, simplifying manufacturing and improving accuracy across different probe configurations.

### CROSS REFERENCE TO RELATED APPLICATIONS

The present application claims priority to U.S. Provisional Patent Application No. 63/778,048, filed Mar. 26, 2025, the disclosure of which is incorporated herein in its entirety.

### TECHNICAL FIELD

Described herein relates generally to temperature measurement in tissue ablation systems and methods of operating such systems. More specifically, the present invention relates to methods and systems for improving temperature measurement accuracy in radiofrequency tissue ablation systems by implementing correction methods that accommodate different thermocouple types while eliminating the need for manufacturing calibration procedures.

### BACKGROUND

Radiofrequency energy has been used to ablate tissue at various locations within the body. For example, radiofrequency probes have been used to ablate tissue within vertebral bodies to treat chronic back pain, to ablate nerves to treat facet joint pain, or to ablate tissue within joints to treat joint pain within the knees, shoulders, elbows, hips, or ankles.

During radiofrequency ablation procedures, accurate temperature measurement is important for ensuring safe and effective therapy delivery. These systems typically utilize thermocouple sensors at the probe tip to monitor tissue temperature. The temperature data guides the surgeon in determining whether sufficient thermal effects have been achieved at the target site.

Traditional radiofrequency generator systems measure temperature using thermocouples that generate a small voltage proportional to the temperature difference between the measuring junction (probe tip) and a reference junction (cold junction). These systems typically employ a single type of thermocouple, most commonly T-type or K-Type, with internal hardware configured specifically for that thermocouple type.

A challenge in these systems is ensuring accurate “Cold Junction” temperature readings across multiple devices and channels. Manufacturing variations, Cold Junction temperature sensor locations on printed circuit boards, and connector interfaces can introduce measurement errors. Traditional systems address these issues through individual device calibration during manufacturing, where each device undergoes a calibration procedure using temperature simulators at specific reference temperatures.

This calibration process adds complexity, time, and cost to manufacturing. Furthermore, the calibration is typically optimized for a single thermocouple type, creating compatibility challenges when different probe types with varying thermocouple materials are introduced. When a system configured for a T-type thermocouple employs a K-type thermocouple, additional calibration and adjustments are necessary, further complicating both manufacturing and clinical use.

Therefore, there is a need to develop methods and systems that can accurately measure temperature with different thermocouple types without requiring individual device calibration during manufacturing. Such universal temperature measurement capabilities would enhance manufacturing efficiency while maintaining or improving temperature measurement accuracy across different device configurations.

### SUMMARY

The present invention addresses the need for accurate temperature measurement in radiofrequency ablation systems by providing a universal method that accommodates different thermocouple types while eliminating the need for manufacturing calibration procedures. The system improves temperature measurement accuracy through a combination of specific corrections to analog-to-digital converter readings and cold junction voltage measurements.

In radiofrequency ablation systems, temperature is typically measured using thermocouples incorporated into the probe tips. Thermocouples operate by generating a voltage based on the temperature difference between two junctions: the measuring junction at the probe tip and the reference junction (cold junction) where the thermocouple wires connect to the standard conductors in the measurement device. To calculate the actual temperature at the probe tip, the system must accurately know the temperature at the cold junction and apply appropriate compensation, which becomes particularly challenging when different thermocouple types are used. For example, at the probe tip there may be two dissimilar metal wires to form a junction, which generate a voltage that could be correlated to temperature changes, allowing for temperature measurement.

The cold junction happens on the opposite end of the tip, when these two dissimilar metal conductors join a measurement circuit (e.g. the instrument, RF ablation system) traces (e.g. copper traces) creating another dissimilar junction. This cold junction voltage is added (or subtracted) in order to have an accurate measurement of temperature.

The system of the present invention provides universal temperature measurement through a series of targeted corrections that address these challenges. The system includes a radiofrequency generator comprising a power source and a controller operatively coupled to the power source and having a processor and a memory element. The radiofrequency generator has multiple connector ports, each associated with a temperature sensor, and an analog-to-digital converter (ADC). Radiofrequency probes with different thermocouple types can be connected to these ports. The ADC converts analog voltage signals from the thermocouple to digital converter voltage signals. The processor executes program instructions that cause the controller to determine the thermocouple type, receive digital converter voltage signals from the ADC, apply a first correction to these signals based on a predetermined correction factor, receive a temperature reading from the temperature sensor associated with the connector port, wherein the temperature reading represents a cold junction voltage signal, apply a second correction to the cold junction voltage signal to compensate for a location offset between the temperature sensor and a cold junction at a connector of the connector port, calculate an adjusted voltage based on the corrected converter voltage signal and the corrected cold junction voltage signal, and convert this to a temperature value using a lookup table corresponding to the determined thermocouple type.

The system implements temperature measurement strategies including applying linear corrections to digital converter voltage signals based on thermocouple type, compensating for the physical distance between cold junction temperature sensors and actual cold junction locations, calculating appropriate temperature voltages by adding corrected cold junction voltages to corrected converter voltage signals, and selecting appropriate conversion tables based on thermocouple type. These capabilities enable the system to accurately measure temperature with different thermocouple types without requiring manufacturing calibration. This universal approach eliminates the need for calibration during manufacturing while enabling accurate temperature measurement regardless of thermocouple type. The system can accommodate both T-type and K-type thermocouples, automatically determining the appropriate correction factors and conversion methods based on the detected thermocouple type. The system thus simplifies manufacturing procedures while improving temperature measurement accuracy.

In Example 1, a temperature measurement system is provided. The temperature measurement system comprises a radiofrequency generator having a power source, a controller operatively coupled to the power source, a processor, a memory element, analog-to-digital converter (ADC), and a plurality of connector ports, wherein each of the plurality of connector ports has at least one temperature sensor associated therewith. The system further comprises a radiofrequency probe electrically coupled to a first one of the plurality of connector ports and having a thermocouple; wherein the ADC is configured to convert an analog voltage signal generated by the thermocouple into a digital converter voltage signal. The processor is configured to execute program instructions to determine a type of thermocouple used in the radiofrequency probe when coupled to the first one of the plurality of connector ports; apply a first voltage correction value to the digital converter voltage signal based on a predetermined correction factor corresponding to the type of thermocouple to generate a corrected converter voltage signal; receive temperature information from the temperature sensor associated with the first one of the plurality of connector ports, the temperature information includes a cold junction voltage signal. The processor further applies a second voltage correction value to the cold junction voltage signal to compensate for a location offset of the temperature sensor relative to the first one of the plurality of connector ports to generate a corrected cold junction voltage signal; determine an adjusted voltage based on the corrected converter voltage signal and the corrected cold junction voltage signal; and convert the adjusted voltage into a temperature value using a conversion technique.

In Example 2, the system of Example 1, wherein the first voltage correction value comprises a linear correction value with coefficient values specific to the type of thermocouple, wherein the linear correction value adjusts the digital converter voltage signal using a formula that includes a slope value multiplied by the digital converter voltage signal plus an intercept value.

In Example 3, the system of Example 2, wherein the second voltage correction value comprises a fixed temperature offset value for adjusting the cold junction voltage signal, wherein the fixed temperature offset value compensates for a physical distance between the temperature sensor and an actual cold junction location at a connector location of the first one of the plurality of connector ports.

In Example 4, the system of any one of Examples 1-3, wherein the first and second voltage correction values eliminate a need for individual radiofrequency probe calibration during manufacturing.

In Example 5, the system of any one of Examples 1-4, wherein the processor is further configured to automatically determine the type of thermocouple used in the radiofrequency probe; and select an appropriate voltage correction value based on the determined thermocouple type.

In Example 6, the system of Examples 5, wherein determining the type of thermocouple comprises detecting one or more characteristics of the radiofrequency probe, wherein the characteristics comprise at least one of electrical resistance measurements, therapy type selections, pin configurations, voltage response patterns, or user interface inputs; and comparing the detected characteristics to predetermined reference values to identify the type of thermocouple.

In Example 7, the system of any one of Examples 1-6, wherein the processor is further configured to determine that the thermocouple is a K-type thermocouple; and select a K-type thermocouple lookup table for converting the adjusted voltage to the temperature value.

In Example 8, the system of any one of Examples 1-7, wherein the processor is further configured to determine that the thermocouple is a T-type thermocouple; and select a T-type thermocouple lookup table for converting the adjusted voltage to the temperature value.

In Example 9, the system of any one of Examples 1-8, wherein the radiofrequency probe comprises a single-use device (SUD) with a K-type thermocouple connected to the connector port via an adapter.

In Example 10, the system of Example 9, wherein the processor is further configured to apply a third voltage correction based on a combination of the SUD and an adapter when calculating the adjusted temperature voltage.

In Example 11, the system of any one of Examples 1-10, wherein the first voltage correction value compensates for electrical and thermal variations introduced by circuit paths between the temperature sensor and a connector location of the first one of the plurality of connector ports.

In Example 12, the system of any one of Examples 1-11, wherein the radiofrequency generator comprises multiple channels, wherein each channel corresponds to one of the plurality of connector ports, and wherein each channel has an independent temperature sensor offset value associated therewith.

In Example, 13, the system of Example 12, wherein the radiofrequency generator comprises four channels.

In Example 14, the system of any one of Examples 1-13, wherein the processor is configured to apply different voltage correction values at different temperatures by selecting specific first and second voltage correction values from a set of predetermined voltage correction values characterized at multiple temperature conditions.

In Example 15, the system of any one of Examples 1-14, wherein the conversion technique comprises using a lookup table or a polynomial conversion function corresponding to the determined thermocouple type.

In Example 16, a temperature measurement system is provided. The temperature measurement system comprises a radiofrequency generator having at least one power source, a controller operatively coupled to the at least one power source, a processor, a memory element, at least one analog-to-digital converter (ADC), and a plurality of connector ports, wherein each of the plurality of connector ports has at least one temperature sensor associated therewith. The system further comprises a radiofrequency probe electrically coupled to a first one of the plurality of connector ports and having a thermocouple; wherein the at least one ADC is configured to convert an analog voltage signal generated by the thermocouple into a digital converter voltage signal. The processor is configured to execute program instructions to determine a type of thermocouple used in the radiofrequency probe when coupled to the first one of the plurality of connector ports; apply a first voltage correction value to the digital converter voltage signal based on a predetermined correction factor corresponding to the type of thermocouple to generate a corrected converter voltage signal; receive temperature information from the at least one temperature sensor associated with the first one of the plurality of connector ports, the temperature information includes a cold junction voltage signal. The processor further applies a second voltage correction value to the cold junction voltage signal to compensate for a location offset of the at least one temperature sensor relative to the first one of the plurality of connector ports to generate a corrected cold junction voltage signal; determine an adjusted voltage based on the corrected converter voltage signal and the corrected cold junction voltage signal; and convert the adjusted voltage into a temperature value using a conversion technique.

In Example 17, the system of Example 16, wherein the first voltage correction value comprises a linear correction value with coefficient values specific to the type of thermocouple, wherein the linear correction value adjusts the digital converter voltage signal using a formula that includes a slope value multiplied by the digital converter voltage signal plus an intercept value.

In Example 18, the system of Example 17, wherein the second voltage correction value comprises a fixed temperature offset value for adjusting the cold junction voltage signal, wherein the fixed temperature offset value compensates for a physical distance between the temperature sensor and an actual cold junction location at a connector location of the first one of the plurality of connector ports.

In Example 19, the system of Example 16, wherein the first and second voltage correction values eliminate a need for individual radiofrequency probe calibration during manufacturing.

In Example 20, the system of Example 16, wherein the processor is further configured to automatically determine the type of thermocouple used in the radiofrequency probe; and select an appropriate voltage correction value based on the determined thermocouple type.

In Example 21, the system of Example 20, wherein determining the type of thermocouple comprises detecting one or more characteristics of the radiofrequency probe, wherein the characteristics comprise at least one of electrical resistance measurements, therapy type selections, pin configurations, voltage response patterns, or user interface inputs; and comparing the detected characteristics to predetermined reference values to identify the type of thermocouple.

In Example 22, the system of Example 16, wherein the processor is further configured to determine that the thermocouple is a K-type thermocouple; and select a K-type thermocouple lookup table for converting the adjusted voltage to the temperature value.

In Example 23, the system of Example 16, wherein the processor is further configured to determine that the thermocouple is a T-type thermocouple; and select a T-type thermocouple lookup table for converting the adjusted voltage to the temperature value.

In Example 24, the system of Example 16, wherein the radiofrequency probe comprises a single-use device (SUD) with a K-type thermocouple connected to the connector port via an adapter.

In Example 25, the system of Example 24, wherein the processor is further configured to apply a third voltage correction based on a combination of the SUD and an adapter when calculating the adjusted temperature voltage.

In Example 26, the system of Example 16, wherein the first voltage correction value compensates for electrical and thermal variations introduced by circuit paths between the temperature sensor and a connector location of the first one of the plurality of connector ports.

In Example 27, the system of Example 16, wherein the radiofrequency generator comprises multiple channels, wherein each channel corresponds to one of the plurality of connector ports, and wherein each channel has an independent temperature sensor offset value associated therewith.

In Example 28, the system of Example 16, wherein the processor is configured to apply different voltage correction values at different temperatures by selecting specific first and second voltage correction values from a set of predetermined voltage correction values characterized at multiple temperature conditions.

In Example 29, the system of Example 16, wherein the conversion technique comprises using a lookup table or a polynomial conversion function corresponding to the determined thermocouple type.

In Example 30, a method of measuring temperature in a tissue ablation system is provided, the tissue ablation system comprises radiofrequency generator having an analog-to-digital converter (ADC) and a plurality of connector ports, wherein each of the plurality of connector ports has associated therewith a temperature sensor, and a radiofrequency probe having a thermocouple that is configured to be coupled to a first one of the plurality of connector ports. The method comprises determining a type of thermocouple used in the radiofrequency probe; converting, by the ADC, an analog voltage signal from the thermocouple into a digital converter voltage signal; applying a first voltage correction value to the digital converter voltage signal to generate a corrected converter voltage signal; generating temperature information from the temperature sensor associated with the first one of the plurality of connector ports that is coupled to the radiofrequency probe, wherein the temperature information includes a cold junction voltage signal; applying a second voltage correction value to the cold junction voltage signal to compensate for a location offset of the temperature sensor relative to the first one of the plurality of connector ports to generate a corrected cold junction voltage signal; calculating an adjusted voltage based on the corrected converter voltage signal and the corrected cold junction voltage signal; and converting the adjusted voltage to a temperature value.

In Example 31, the method of Example 30, wherein the first voltage correction comprise linear corrections with coefficient values specific to the thermocouple types, wherein the linear corrections adjust the digital converter voltage signals using formulas comprising slope values multiplied by the digital converter voltage signals plus intercept values.

In Example 32, the method of Example 30, wherein the second voltage correction value comprises a fixed temperature offset value for adjusting the cold junction voltage signal, wherein the fixed temperature offset value compensates for a physical distance between the temperature sensor and an actual cold junction location at a connector location of the first one of the plurality of connector ports.

In Example 33, the method of Example 30, further comprising automatically determining, by a processor of the generator, the type of thermocouple used in the radiofrequency probe; and selecting an appropriate voltage correction value based on the determined thermocouple type.

In Example 34, the method of Example 30, wherein determining the type of thermocouple comprises detecting, by the processor, one or more characteristics of the radiofrequency probe, wherein the one or more characteristics comprise at least one of electrical resistance measurements, therapy type selections, pin configurations, voltage response patterns, or user interface inputs; and identifying, by the processor, the type of thermocouple by comparing the detected characteristics to predetermined reference values stored in a memory of the radiofrequency generator.

In Example 35, the method of Example 30, wherein the conversion of the adjusted voltage to the temperature value is done using a look-up table or a polynomial conversion function corresponding to the determined thermocouple type.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

Having thus described embodiments of the present disclosure in general terms, reference will now be made to the accompanying drawings, which are not necessarily drawn to scale, and wherein:

FIG. 1 is a schematic block diagram of an exemplary multichannel radiofrequency tissue ablation system, in accordance with the teachings of the present invention;

FIG. 2 is a schematic flow chart diagram illustrating a conventional approach to temperature measurement in a tissue ablation system using T-type thermocouple calibration, in accordance with the teachings of the present invention;

FIG. 3 is a schematic flow chart diagram illustrating temperature measurement in a tissue ablation system with correction for different thermocouple types, in accordance with the teachings of the present invention;

FIG. 4 is a schematic block diagram illustrating a K-type Single Use Device (SUD) with adaptor thermal couple characterization configuration, in accordance with the teachings of the present invention;

FIG. 5 is a schematic block diagram illustrating a T-type thermal couple characterization configuration, in accordance with the teachings of the present invention;

FIG. 6 is a schematic block diagram illustrating a SUD/K-type wiring thermal couple characterization configuration, in accordance with the teachings of the present invention; and

FIG. 7 is a graph illustrating ADC adjustment with a linear correction formula, in accordance with the teachings of the present invention.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure. Throughout this section it should be understood that the temperature measurement system processes thermocouple signals in the voltage domain until final conversion to temperature values. Thermocouples generate voltage signals proportional to temperature differences, and the system applies correction to these voltages. While various temperature sensors provide readings, these readings are represented and processed as voltage signals, references to temperature measurements and cold junction temperatures should be understood in this context.

A radiofrequency ablation (RFA) procedure is a minimally invasive medical procedure that is used to ablate targeted tissue at a surgical site, and typically involves using one or more energy delivery devices (e.g., radiofrequency (RF) probes) having one or more electrodes associated therewith. The radiofrequency probe can be coupled to one or more radiofrequency (RF) generators to deliver power in the form of radiofrequency energy to the targeted tissue via the electrodes. The radiofrequency energy generates localized heat which is used to target and destroy (e.g., ablate) the targeted tissue. In pain management, the targeted tissue can be nerve tissue responsible for sending pain signals.

The RFA procedure can be utilized to ablate different types of tissue, such as nerve tissue, adipose tissue, muscle tissue, and the like. The RFA procedure can be performed at various locations on an external surface of a subject or patient or within the subject. The RFA procedure performed within the subject can be performed in a minimally invasive manner (e.g., via a percutaneous, laparoscopic, endoscopic, or intravascular approach) or in a more invasive manner (e.g., via an open surgical procedure). For example, Applicant's existing technology (the Intracept® procedure by Relievant® Medsystems, Inc.) offers a safe and effective minimally invasive procedure that targets the basivertebral nerve (BVN) for the relief of chronic low back pain that originates at least partly from one or more endplates of one or more vertebral bodies. The Intracept® procedure involves application of radiofrequency energy from a radiofrequency generator using a bipolar radiofrequency probe, where the applied radiofrequency energy is sufficient to ablate the basivertebral nerve within the vertebral body. The procedure can be performed in multiple different vertebral bodies sequentially or simultaneously using a single radiofrequency probe or using multiple radiofrequency probes coupled to a single generator. The basivertebral nerve trunk can then be ablated by the RF probe. Other intraosseous nerves within the vertebral body that innervate the endplates and/or intervertebral disc can also be targeted and ablated.

The RFA procedure is typically performed using a surgical introducing or access assembly that can include a hollow cannula that forms a sheath and a stylet that is disposed within the cannula until a distal tip of the stylet extends beyond the cannula. The stylet adds rigidity and support to the cannula when inserted into the patient. The stylet helps the surgeon guide the assembly to the surgical site. Once the cannula is in position at the surgical site, then the stylet is removed from the cannula and the RF probe is inserted therein. The RF probe can then deliver RF energy to the targeted tissue at the surgical site. In a bipolar RF probe arrangement, the probe can include a pair of electrodes, including a first active energy delivering electrode and a second return electrode. According to another arrangement, the RF probes can be configured in a monopolar arrangement, where an RF probe employs an active energy delivering electrode and a separate device or element can be used as a return. The return can be configured as a return pad coupled to the subject or a return electrode disposed on a separate RF probe. For example, a first RF probe can be used that includes the active energy delivering electrode and a second RF probe can be used that includes a return electrode. The RF probe can also include additional structure, including for example temperature sensors (e.g., thermocouples or thermistors), that can be employed to sense or detect the temperature at the surgical site during the surgical procedure. The sensed or detected temperature can provide important information to the surgeon about the ablation procedure. The RFA procedure contemplates positioning the two electrodes of the one or more radiofrequency probes within the vertebral body. The RF generator supplies power to the active electrode of the RF probe at a selected power or voltage level and for a time sufficient to create a desired lesion within the vertebral body sufficient to ablate the basivertebral nerve within the vertebral body. In some implementations, the step of applying power to the RF probe includes supplying power for a selected duration or period of time (e.g., 1 minute to 1 hour or for any time within this range) to achieve a selected temperature at the surgical site. The temperature of the thermal energy supplied by the RF probe to the targeted tissue can range from between about 50° C. and about 115° C. (e.g., from about 70° C. to about 90° C., from about 75° C. to about 90° C., from about 83° C. to about 87° C., from about 80° C. to about 100° C., from about 85° C. to about 95° C., from about 90° C. to about 110° C., from about 95° C. to about 115° C., or overlapping ranges thereof). The temperature ramp-up during the surgical procedure can range from between about 0.1-5 degrees Celsius/second (e.g., 0.1-1.0 degrees Celsius/second, 0.25 to 2.5 degrees Celsius/second, 0.5-2.0 degrees Celsius/second, 1.0-3.0 degrees Celsius/second, 1.5-4.0 degree Celsius/second, 2.0-5.0 degrees Celsius/second). The time of treatment can range from between about 10 seconds and about 1 hour (e.g., from 10 seconds to 1 minute, 1 minute to 5 minutes, from 5 minutes to 10 minutes, from 5 minutes to 20 minutes, from 8 minutes to 15 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy can be delivered as an alternative to or in sequence with continuous energy. For radiofrequency energy, the energy applied by the RF generator can range from between about 350 kHz and about 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 kHz±5 kHz, 475 kHz±5 kHz, 487 kHz±5 kHz). The power of the radiofrequency energy generated by the RF generator can range from between about 5 W and about 30 W (e.g., from 5 W to 15 W, from 5 W to 20 W, from 8 W to 12 W, from 10 W to 25 W, from 15 W to 25 W, from 20 W to 30 W, from 8 W to 24 W, and overlapping ranges thereof, or any value within the recited ranges).

Although described primarily in connection with procedures for the treatment within the spine, the systems and methods of the present invention can also be used for radiofrequency tissue ablation procedures intended to treat ailments or conditions other than those associated with the spine or back pain. The procedures can involve ablation of nerves outside of bones but related to the spine (e.g., sacroiliac joints, facet joints, etc.). Target treatment locations within bones other than vertebral bodies may also be accessed. For example, target treatment locations within a humerus, radius, femur, tibia, calcaneus, tarsal bones, hips, knees, phalanges, and/or other orthopedic targets can be accessed. The ablation procedures can include, for example, ablation of nerves within or surrounding other bones other than the vertebral column, cardiac tissue ablation for treatment of atrial fibrillation or other abnormal heart rhythms irregularities, tumor ablation at any location within the body (e.g., within bones, lungs, breasts, thyroids, livers, or other organs or tissues), peripheral nerve ablation, pulmonary artery ablation, renal denervation procedures, uterine fibroid ablation, endometrial ablation, and/or the like. The systems and methods described herein may be used in connection with any radiofrequency procedure during which impedance (e.g., tissue impedance or impedance between two electrodes) is monitored.

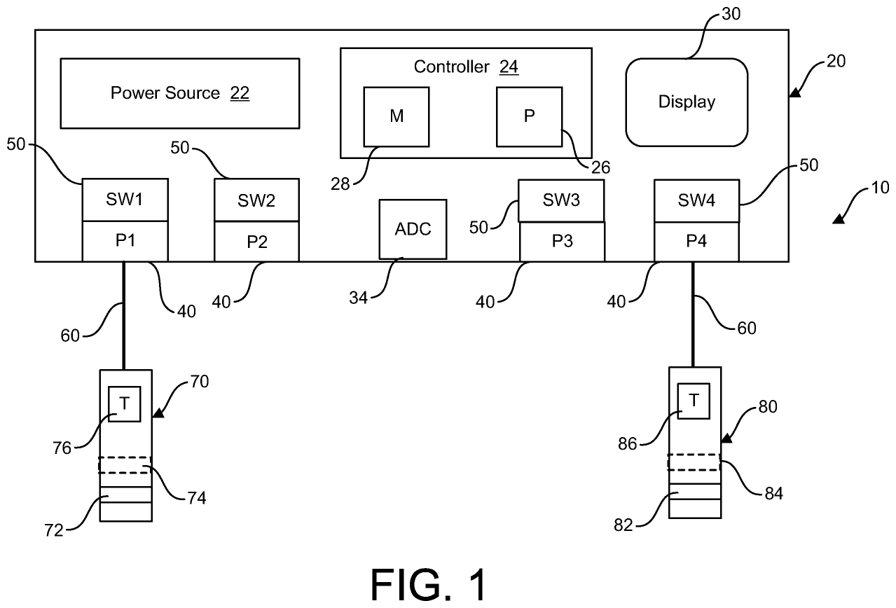

FIG. 1 is a schematic block diagram of a tissue ablation system 10 suitable for use with the present invention. The tissue ablation system includes a generator, such as a radiofrequency generator 20, that is coupled to one or more radiofrequency probes 70, 80. The RF generator 20 can be electrically and communicatively coupled to the RF probes 70, 80 via any suitable conduit 60. The conduits 60 can facilitate bidirectional communication of electrical signals and data or instructions between the RF generator 20 and the RF probes 70, 80. For example, the conduits 60 can include one or more electrical wires or lines (not shown) suitable for conveying the electrical signals. The generator can also be any other type of energy source suitable for supplying ablative energy to the probes. According to one embodiment, the illustrated RF generator 20 can include a power source 22 that can be configured to generate and supply radiofrequency power or energy to the RF probes 70, 80 via the conduits 60 at a desired frequency and power level. In some embodiments, there may be one or more power source. The RF generator 20 can also include a controller 24 that includes for example a processor (e.g., CPU) 26 suitable for processing and executing applications and instructions that are stored in a memory element 28. The RF generator 20 can also include an analog-to-digital converter (ADC) 34 that converts analog voltage signals generated by the thermocouples in the RF probes 70, 80 into digital voltage values that can be processed by the processor 26 of the controller 24. The ADC 34 can be configured to generate converter voltage signals having an associated value based on the received thermocouple voltage signals. The RF generator 20 may also comprise internal temperature sensors (not shown) positioned near the connector ports 40, which measure temperatures associated with the cold junction locations for the thermocouples in the radiofrequency probes. The processor 26 can be any suitable type of processor, such as a special purpose processor, and the memory element 28 can be any suitable type of memory. The processor 26 can be implemented in hardware, software, firmware, or any suitable combination thereof, and can be configured to execute program instructions stored in the memory element 28. The processor 26 and the memory element 28 are shown herein as forming part of the controller 24, but one of ordinary skill in the art will readily recognize that the processor 26 and the memory 28 can form part of other components of the RF generator 20. For the sake of simplicity, only one processor 26 and memory element 28 are shown, although multiple processors and memory elements can also be employed in the RF generator 20. The controller 24 can be configured as described herein, and at least is configured to monitor temperature, power, and/or impedance in connection with a tissue ablation procedure performed by the tissue ablation system 10. The controller 24 may be specifically configured to apply corrections to thermocouple readings based on the thermocouple type used in the radiofrequency probes, including separate corrections for the ADC readings and cold junction voltage measurements. The RF generator 20 can further include a user interface or display element 30 suitable for displaying information or instructions to a user (e.g., surgeon) via a series of user interfaces or for allowing the user to provide instructions to the RF generator. As such, the display element 30 can employ a user interface generator 32 for generating the user interfaces based on programmatic instructions stored in the memory element 28 and processed by the processor 28 or by a processor forming part of the display element 30. The display element 30 can be configured to display information to the user. For example, during startup and use, the current status of the RF generator 20 and energy delivery or treatment parameters can be displayed on the display element 30 via one or more user interfaces. During energy delivery, the display element 30 can be configured to display desired treatment time, remaining treatment time, temperature, impedance, and power information (alphanumerically and/or graphically). For example, graphical representations of power vs. time and impedance vs. time can be displayed. The display element 30 can also be separate from the RF generator 20 and need not form part of the generator. Although not shown, the tissue ablation system 10 can also employ additional accessory devices, such as suitable user input devices including a keyboard, mouse, trackpad, voice-activated input device, and the like.

The illustrated RF generator 20 can also have formed therein a plurality or series of connector ports 40 that allow the RF probes 70, 80 to be coupled to the generator. The RF generator 20 can have any selected number of connector ports 40. In the illustrated embodiment, the RF generator 20 includes four probe connector ports 40A and a ground connector port 40B. The connector ports 40 can have any selected size, shape, and configuration. According to one embodiment, the connector ports 40 can employ a series of electrical pins that connect to RF probes and a ground pad and allow electrical energy and data to be exchanged therebetween. For example, a first electrical pin (or multiple electrical pins) can be coupled to the RF probe so as to deliver RF power to an energy delivering electrode when placed thereon and a second electrical pin can communicate with a return electrode when placed thereon. A third electrical pin can be employed to communicate with a temperature sensor in the RF probe and a fourth electrical pin can be used to exchange data between the RF generator and the RF probe. Those of ordinary skill in the art will readily recognize that any suitable type (e.g., Lemo or Din type connectors), arrangement, and number of electrical connectors can be employed in the connector ports 40.

The illustrated RF generator 20 can also employ a switching element module or a series of discrete switching elements 50 that can be used to help control the generation, modulation, and delivery of RF energy. The switching elements 50 help ensure the precise delivery of energy to the target tissue via the RF probes while maintaining system safety and functionality. The switching elements 50 can also control the RF energy delivered to the active energy delivering electrode of the RF probe through an associated connector port 40. For the sake of simplicity, a switching element 50 is shown coupled to and disposed in electrical communication with each illustrated connector port 40. Those of ordinary skill in the art will readily recognize that a separate switching element module that is arranged in electrical communication with each of the connector ports 40 can also be employed. The illustrated switching elements 50 can also be positioned and arranged to manage the activation of circuits connected to sensors in the RF probes 70, 80, such as temperature sensors or impedance measurement systems, and to manage the activation of the ground connector port 40B. The switching elements 50 can also be arranged to route sensor signals to specific monitoring circuits in the controller 24 for processing. The switching elements 50 can also serve to verify proper grounding or return electrode connection before enabling RF output to ensure patient safety. The switching elements 50 can be any suitable type of switching element, such as MOSFETs, IGBTs, Pin diodes, mechanical and solid-state relays, and the like.

The illustrated tissue ablation system 10 can also employ one or more RF probes. For the sake of simplicity and for purposes of explanation, a pair of RF probes 70, 80 are shown connected to the RF generator 20. The RF probes 70, 80 can be the same or can be different depending upon the selected type of surgical procedure being performed and/or the purpose and function of the RF probe. The RF probe 70 has a main body having an outer surface having one or more electrodes mounted thereon. In a bipolar RF probe arrangement, the RF probe 70 can have a first active energy delivering electrode 72 and a return electrode 74 disposed on an outer surface of the main body. The probe can also include a temperature sensor 76 for sensing or measuring temperature at the surgical site. The electrodes and the temperature sensor 76 can be coupled to the RF generator 20 via electrical leads that pass through the conduit 60. If the RF probe 70 is constructed as a monopolar device, then the probe can include a single active electrode 72, which can function as the active energy delivering electrode or as the return electrode. In this type of arrangement, the second electrode 74 (in dashed lines) is either inactivated or not present. Similarly, the RF probe 80 has a main body having an outer surface having one or more electrodes mounted thereon. In a bipolar RF probe arrangement, the RF probe 80 can also have a first active energy delivering electrode 82 and a return electrode 84. The probe can also include a temperature sensor 86. The electrodes and the sensor can also be coupled to the RF generator 20 via electrical leads that pass through the conduit 60. If the RF probe 80 is constructed as a monopolar device, then the probe can also include a single active electrode 82, which can function as the active energy delivering electrode or as the return electrode. In this type of arrangement, the second electrode 84 is either inactivated or not present. Each RF probe that is coupled to a connector port 40 of the RF generator 20 forms in essence a channel. As such, multiple RF probes coupled to multiple connector ports 40 of the RF generator 20 can form multiple channels.

The systems and methods described herein are directed to improving temperature measurement accuracy in radiofrequency ablation systems while eliminating manufacturing calibration procedures. The system accommodates different thermocouple types in radiofrequency probes 70 and 80 through a universal measurement approach that applies specific corrections to analog-to-digital converter readings and cold junction voltage measurements. The radiofrequency generator 20 includes temperature sensors positioned near the connector ports 40 that measure temperatures associated with the cold junctions where thermocouple wires connect to the standard copper conductors on the circuit board. In some embodiments, one or more temperature sensors may be positioned. These temperature sensors are positioned at distances from the actual cold junctions, creating offsets that are compensated for in the measurement process.

The controller 24 applies a first correction to the analog-to-digital converter (ADC) reading from the thermocouple in the radiofrequency probe. This correction takes the form of a linear formula with coefficients specific to the thermocouple type, compensating for variations introduced by the signal path and material transitions. The controller 24 also applies a second correction to the measured cold junction voltage to compensate for the physical distance between the temperature sensor and the actual cold junction at the connector port.

After applying both corrections, the controller 24 calculates an adjusted temperature voltage and converts this to a temperature value using a lookup table corresponding to the thermocouple type. The system can be configured to automatically detect the thermocouple type or enable manual selection through the user interface 30. This approach eliminates the need for individual device calibration during manufacturing while maintaining accurate temperature measurement across different thermocouple types and connection configurations.

The radiofrequency generator 20 features a multi-channel design, with each channel having its own connector port and associated temperature sensor. Each channel operates independently and has channel-specific correction values, enabling simultaneous connection of multiple probes that may use different thermocouple types. The universal temperature measurement approach applies the appropriate corrections to each channel based on the thermocouple type and channel configuration, ensuring accurate temperature measurement across all channels without requiring manufacturing calibration.

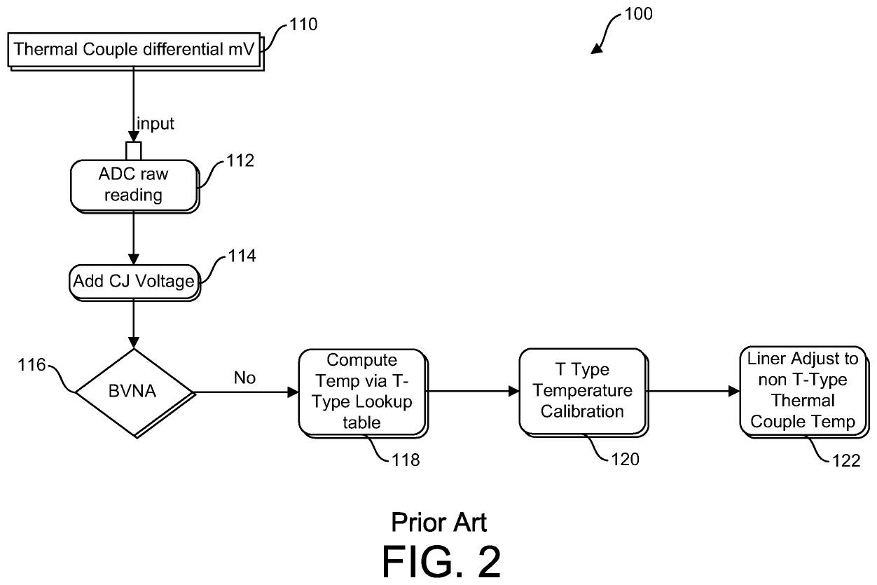

FIG. 2 illustrates a schematic flow chart diagram 100 depicting a conventional approach to temperature measurement in a tissue ablation system. This conventional approach has been used in the Applicant's existing technology (the GX1 system) for measuring temperature in radiofrequency ablation procedures. The flow chart 100 outlines the sequence of steps and components involved in processing temperature data from a thermocouple to a usable temperature value.

A thermocouple, as mentioned, is a temperature sensor consisting of two dissimilar metal wires joined at one end, which produces a small voltage proportional to the temperature difference between the temperature at the joined end (e.g., measuring junction) and an opposite end (e.g., reference or cold junction) that is coupled to the RF generator 20. In radiofrequency ablation systems, thermocouples are integrated into the probes 70, 80 to measure tissue temperature during procedures.

The illustrated flow chart diagram 100 begins with a thermocouple differential input as measured in millivolts (mV). The thermocouple differential input represents the voltage generated by the thermocouple in the radiofrequency probe when the probe tip contacts tissue. This voltage signal varies based on the temperature at the probe tip relative to the cold junction voltage.

The cold junction in a thermocouple circuit refers specifically to the point where the thermocouple wires connect to standard copper conductors on the circuit board in the RG generator 20. At this junction, a transition occurs between the thermocouple materials and the copper traces on the printed circuit board. This junction creates a secondary thermoelectric effect that needs to be compensated for when calculating probe temperature. Accurate measurement of the cold junction voltage is necessary for proper thermocouple operation, as the voltage generated by a thermocouple represents the temperature difference between the measuring junction (at the probe tip) and the reference or cold junction.

From the thermocouple differential input, step 110, the voltage signal flows to an analog-to-digital converter (ADC) which produces an ADC raw reading, step 112. The analog-to-digital converter converts the continuous analog voltage signal from the thermocouple into a discrete digital signal that can be processed by the system's digital circuits and microprocessors. The ADC raw reading can be represented as a digital value corresponding to the analog voltage level detected from the thermocouple.

After digitization, the controller 24 of the RF generator 20 proceeds to add the cold junction (CJ) voltage, step 114. Because the thermocouples 76, 86 measure temperature differences rather than absolute temperatures, the controller 24 needs to account for the temperature at the cold junction point to calculate the actual temperature at the probe tip.

The cold junction voltage is derived from temperature sensors within the radiofrequency generator 20 that are positioned near the connector ports 40. These temperature sensors measure the temperature near the cold junction point, which is then converted to an equivalent voltage value. This voltage value is added to the ADC raw reading to compensate for the reference junction temperature.

Following the cold junction compensation, the controller 24 reaches a decision point, step 116, concerning a specific therapy mode or operating condition. This decision point determines the next step in the processing methodology based on the type of therapy being delivered or the configuration of the connected radiofrequency probe.

At the decision point, if the type of therapy evaluates is not a BVN therapy (e.g., “No”), then the controller 24 determines the probe temperature using a T-type specific measurement methodology. T-type thermocouples are commonly used in the probes 70, 80 due to their accuracy in the physiological temperature range. At the decision point, if the condition evaluates to “Yes,” the system follows a different determination methodology (not shown in FIG. 2) for other therapy modes or special conditions. In the conventional approach, the “Yes” path may typically lead to alternative temperature calculation methods or bypasses temperature monitoring entirely depending on the specific therapy being delivered.

In the illustrated flowchart 100, the controller 24 computes the probe temperature by referencing a prestored T-type lookup table, step 118. The lookup table is a data structure that contains pre-calculated conversion voltage values that map to corresponding temperatures. The T-type lookup table can include standardized conversion factors specific to T-type thermocouples, which relate the compensated voltage readings to specific temperature values based on the known characteristics of T-type thermocouple materials. The lookup table approach is used because the relationship between thermocouple voltage and temperature is non-linear. Rather than performing complex calculations for each measurement, the controller 24 can reference a table of pre-calculated voltage values to determine the temperature based on the measured voltage.

After the temperature computation through the T-type lookup table, the conventional approach includes a T-type temperature calibration, step 120. The calibration step is a device-specific adjustment that accounts for manufacturing variations and component tolerances in each individual radiofrequency generator.

During the manufacturing process of conventional systems, each radiofrequency generator 20 undergoes calibration where temperature measurements are taken at known reference points, which may typically be at 40° C. and 90° C. The differences between the measured values and the known reference temperatures are used to calculate calibration factors, which are then stored in the memory 28 and applied during calibration, step 120.

The final step in the conventional temperature determination approach involves applying a linear adjustment to the non-T-type thermal couple temperature, step 122. This adjustment is applied when the system is used with thermocouples other than T-type, such as K-type thermocouples. The linear adjustment applies a mathematical correction to the calibrated T-type temperature to accommodate for the different voltage-temperature characteristics of other thermocouple types.

The linear adjustment typically takes the form of a simple linear equation with a slope and intercept value that converts the T-type calibrated temperature to an adjusted temperature appropriate for the connected thermocouple type. While this approach functions adequately, it relies on the underlying T-type calibration and does not directly address the fundamental differences in thermocouple characteristics.

As conventional generators may be optimized primarily for T-type thermocouples, it provides less accurate results when using other thermocouple types, such as K-type thermocouples, which are composed of different material and have different voltage-temperature characteristics. Furthermore, the conventional approach does not directly compensate for the physical distance between the temperature sensors within the radiofrequency generator and the actual cold junction locations at the connectors. This physical separation introduces a temperature offset that affects measurement accuracy.

These limitations of the conventional generators employing the conventional temperature calculation methodology require individual device calibration during manufacturing. This increases production time and costs associated with the RF generator. As a result, the development of a universal temperature measurement methodology as described herein is necessary. The universal approach of the present invention addresses the foregoing challenges through specific temperature calculation corrections for both the ADC readings and the cold junction voltages, eliminating the need for manufacturing calibration while improving temperature calculation accuracy for different thermocouple types.

More specifically, the conventional approach shown in FIG. 2 requires a calibration procedure during manufacturing where each individual radiofrequency generator is calibrated using temperature simulators. During this procedure, known temperatures are applied to the system, measurements are taken, and device-specific calibration factors are calculated and stored in memory. This calibration step adds time, complexity, and cost to the manufacturing process. Each RF generator needs to be individually assessed and characterized, and the calibration factors that are calculated are stored and maintained within the generator. The universal temperature measurement approach of the present invention eliminates this manufacturing calibration requirement by applying predetermined correction factors based on thermocouple type and channel configuration, significantly simplifying the manufacturing process while maintaining or improving measurement accuracy.

For example, the conventional calibration procedure involves testing each of the four ports 40 individually using a T-Type temperature simulator. The simulator is set to two specific temperature points: 40° C. and 90° C. At each temperature point, the radiofrequency generator's temperature reading is recorded. Based on the differences between the known simulator temperatures and the measured values, a linear correction function is calculated for each port. These individual correction functions are then stored in the generator's memory and applied during normal operation. This calibration procedure is repeated for every device during manufacturing, adding time and cost to the production process. In contrast, the universal temperature measurement approach eliminates this device-specific calibration step by applying predetermined correction factors based on thermocouple type and channel configuration.

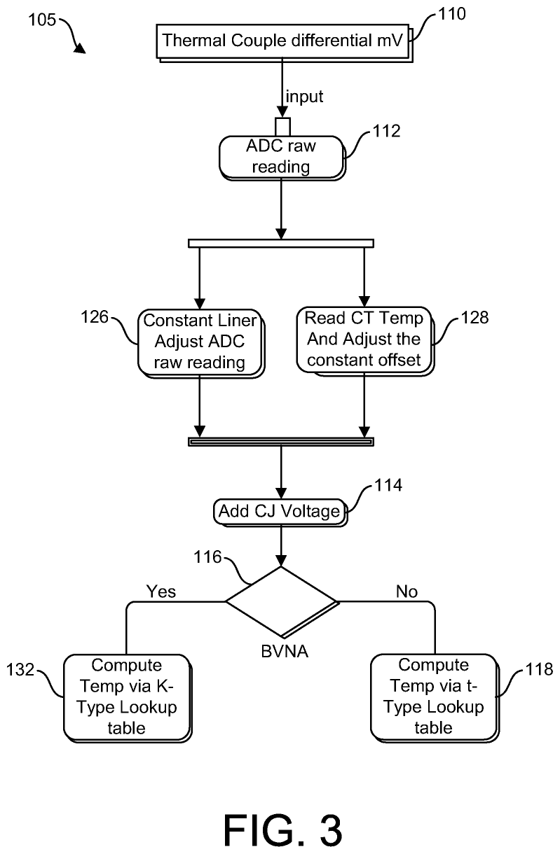

The limitations of the conventional approach highlighted the need for a more versatile and efficient method of temperature measurement that could accommodate different thermocouple types without requiring individual device calibration. FIG. 3 illustrates the improved approach to temperature measurement developed in the present invention.

FIG. 3 illustrates a schematic flow chart diagram 105 depicting an improved approach to probe temperature measurement in a tissue ablation system. This improved approach eliminates the need for manufacturing calibration of the RF generator 20 while providing accurate temperature measurements for different types of thermocouples. The flow chart diagram 105 outlines the sequence of steps associated with determining probe temperature and the associated components involved in the universal temperature measurement method.

Thermocouples are temperature sensors that generate small voltages proportional to temperature differences. Different thermocouple types have distinct voltage-temperature characteristics and operating ranges. T-type thermocouples are commonly used in medical devices due to their accuracy in the physiological temperature range and stability. K-type thermocouples offer a wider temperature range and different sensitivity characteristics. The RF generator 20 employs includes connector ports 40 that can employ an internal T-type wiring architecture. The universal temperature measurement technique of the present invention enables accurate temperature measurements and readings with different thermocouple types by applying specific corrections based on the thermocouple type used in the radiofrequency probe 70, 80.

A central challenge in thermocouple temperature measurement arises when there are material transitions or mismatches in the thermocouple circuit. When different metals are introduced in the signal path, such as connecting a K-type thermocouple to a generator with T-type internal wiring, the material transitions create additional voltage offsets that affect temperature measurement accuracy. The universal temperature measurement technique addresses these material mismatches by employing predetermined correction factors that account for the specific voltage deviations introduced by each transition point in the circuit.

Similar to the conventional approach, the flow chart 105 begins with a differential voltage reading from a thermocouple, step 110. The voltage reading can be mV. The thermocouple differential reading represents the voltage generated by the thermocouple in the radiofrequency probe 70, 80 when the probe tip contacts tissue. The voltage signal varies based on the temperature at the probe tip relative to the cold junction voltage in the RF generator 20, and the magnitude of the voltage varies depending on whether a T-type or K-type thermocouple is being used.

The differential voltage signal from the thermocouple is then processed by an analog-to-digital converter, which generates an ADC raw output reading, step 112. The ADC raw reading is a digital representation of the analog voltage signal from the thermocouple, enabling the controller 24 to perform selected types of calculations based on the voltage data.

Unlike the conventional approach, the method of the present invention performs adjustments to the ADC raw readings prior to the addition of the cold junction voltage. For example, the universal temperature measurement technique contemplates applying a first voltage adjustment and a cold junction voltage adjustment to the voltage readings from the converter.

Specifically, the method of the present invention contemplates applying an adjustment or correction value, such as a constant linear adjustment or correction value, to the ADC raw voltage reading, step 126, based on predetermined correction factors specific to the type of thermocouple being used. This first voltage correction takes the form of a linear formula that adjusts the ADC reading using the equation y=mx+b, where y is the corrected ADC value, x is the raw ADC reading, m is a slope coefficient, and b is an intercept value. The slope and intercept values are determined through characterization of multiple devices to establish consistent correction factors or values for each thermocouple type.

The first correction values differ for T-type and K-type thermocouples due to their different voltage-temperature characteristics. Additionally, when K-type thermocouples are connected to a system with T-type internal wiring, material transitions create additional voltage offsets that must be compensated for. The linear correction formula accounts for these differences by applying thermocouple-specific coefficients.

The first correction value compensates for variations in the ADC readings due to the specific characteristics of different thermocouple types and the signal paths within the radiofrequency generator. The linear correction formula transforms the raw ADC values to correspond more closely to the actual thermocouple voltage, accounting for any systematic errors or offsets introduced by the analog-to-digital conversion process. In some embodiments, the system can apply specific slope and intercept values derived from characterization data across multiple devices, ensuring consistent performance without requiring individual device calibration.

Simultaneously, the system obtains a temperature reading from the temperature sensor associated with the connector port, step 128. This temperature reading represents the cold junction voltage near the connector where the thermocouple wires meet the standard copper conductors on the circuit board.

The system applies a second correction value or factor to the cold junction voltage to compensate for the location offset between the temperature sensor and the actual cold junction at the connector. This correction addresses the physical distance between where the temperature is measured and where the actual cold junction exists. In the radiofrequency generator 20, multiple channels are provided to connect different probes simultaneously. Each channel has its own connector port 40 and associated temperature sensor. Due to the internal layout of the radiofrequency generator 20, the temperature sensors are positioned at varying distances from their respective connector ports, creating channel-specific temperature offsets. The second correction value thus applies channel-specific offset values determined through thermal characterization of the radiofrequency generator design. This enables each channel to have its own calibrated temperature correction, ensuring accurate readings across all channels without requiring individual device calibration during manufacturing.

The radiofrequency generator features a multi-channel design with four separate measurement channels, each with its own connector port and associated temperature sensor. Each channel operates independently, enabling simultaneous connection of multiple probes that may use different thermocouple types. In this multi-channel configuration, each channel requires its own specific cold junction voltage correction, as the physical layout of the device creates different offset distances between each temperature sensor and its corresponding connector. The universal temperature measurement approach applies channel-specific correction values, such as different offset values for Channel 1 versus Channel 4, ensuring accurate temperature measurement across all channels without requiring individual calibration during manufacturing.

After applying the first correction value to the ADC reading and the second correction value to the cold junction voltage, the system then adds (or subtracts) the CJ Voltage, step 114. This step calculates an adjusted temperature voltage based on the corrected ADC reading and the corrected cold junction voltage, providing a more accurate representation of the actual temperature at the probe tip.

Following the calculation of the adjusted voltage, the controller 24 then determines if a BVNA procedure is being performed, which is indicative of the type of probe to be used, step 116. This controller 24 determines based on this information which lookup table to use for converting the adjusted voltage to a temperature value based on the thermocouple type, such as a K-type thermocouple and a T-type thermocouple.

If a BVNA procedure is being performed, step 132, then a K-type thermocouple is being used, and the controller 24 proceeds to determine the temperature using a K-Type lookup table. The lookup table can include standardized conversion factors specific to K-type thermocouples, which correlate the adjusted voltage readings to specific temperature values based on the known characteristics of K-type thermocouple materials.

If the BVNA procedure is not being performed, step 118, then the controller 24 determines the temperature using a T-type look-up table since a T-type thermocouple is in use. The T-type lookup table includes standardized conversion factors specific to T-type thermocouples, allowing accurate temperature conversion.

The lookup tables for both thermocouple types include voltage-to-temperature conversion values based on standard reference functions for each thermocouple type. These functions can be established by organizations and provide standardized conversion between thermocouple voltage and temperature. By selecting the appropriate lookup table based on the thermocouple type, the RF generator 20 ensures accurate temperature conversion regardless of the thermocouple type being used.

The universal temperature measurement technique employed by the RF generator 20 can automatically detect the type of thermocouple connected to each channel. The probe detection can be implemented through various methods, including electrical resistance measurements, pin configurations, voltage response analysis, therapy type selections, or user interface inputs. When a probe is connected to a port 40, the processor 26 can analyze electrical characteristics associated with the probe and port to determine whether a T-type thermocouple or a K-type thermocouple has been connected thereto, comparing the detected characteristics to predetermined reference values stored in memory, and then automatically select the appropriate voltage correction factors and values via lookup tables for that specific probe type. The processor 26 may utilize dedicated sensing circuitry within the RF generator 20 to measure electrical resistance or voltage responses, or it may read digital signals from pin configuration detectors at the connector ports. For example, when a procedure is selected by the user, the processor may automatically determine that a K-type thermocouple is being used. Similarly, specific pin configurations at the connector interface or distinctive voltage response patterns may indicate whether a T-type or K-type thermocouple is connected. This automatic detection capability enhances system usability by eliminating the need for manual configuration when different probe types are used, while ensuring that the correct measurement parameters are applied for each probe.

The voltage adjustment and correction technique illustrated in FIG. 3 eliminates the need for the T-type temperature calibration step that was required in the conventional approach. By applying specific corrections to both the ADC reading and the cold junction voltage, the system achieves accurate temperature measurements without requiring individual device calibration during manufacturing.

Additionally, the voltage adjustment and correction technique directly accommodates different thermocouple types by applying probe type-specific correction factors and using appropriate probe specific lookup tables. This versatility enables the system to work with both T-type and K-type thermocouples without compromising accuracy, providing a universal temperature measurement technique that simplifies manufacturing while improving performance.

In some embodiments the voltage signal may undergo comprehensive signal conditioning before analog-to-digital conversion. A thermocouple generates a direct current (DC) voltage that is proportional to the temperature difference between two junctions, typically producing a small millivolt-range signal that varies predictably based on the specific metals used. To ensure accurate temperature measurement, the system may also incorporate one or more low-pass filters to isolate this pure DC thermocouple voltage. This filter may eliminate high-frequency noise and electromagnetic interference that could compromise measurement precision. These low-pass filters are may be followed by differential amplifiers that provide voltage amplification and noise rejection, enabling the extraction of the DC voltage signal. The signal path may typically follow a configuration of low-pass filter>differential amplifier>analog-to-digital converter (ADC), ensuring clean, stable, and accurate thermocouple voltage measurements. This signal conditioning approach may further enhance the system's ability to extract reliable temperature information across various thermocouple types and operating conditions, complementing the universal temperature measurement technique described herein.

In some embodiments, the temperature conversion process may employ a polynomial conversion function instead of or in addition to the standard lookup table. The polynomial conversion function provides a mathematical approach to converting the adjusted thermocouple voltage to a temperature value, utilizing a polynomial equation that captures the non-linear voltage-temperature relationship inherent in thermocouple materials. For example, a typical polynomial conversion function for a thermocouple may take the form:
T=a0+a1V+a2V²+a3V³+a4V⁴+ . . . +anVⁿ

Where: T represents the temperature, V represents the measured thermocouple voltage, a0, a1, a2, etc., are polynomial coefficients specific to the thermocouple type, and n represents the order of the polynomial.

For instance, a T-type thermocouple and a K-type thermocouple may have different polynomial coefficients that reflect their material characteristics. The K-type thermocouple polynomial, for example, has different coefficient values to accurately convert voltage to temperature compared to the T-type thermocouple.

This polynomial approach provides a continuous interpolation across the entire temperature range, eliminating the discrete steps inherent in lookup table-based conversions. The coefficients can be derived through characterization of the thermocouple type, accounting for variations in the voltage-temperature relationship. Furthermore, the polynomial method enables for more flexible mathematical manipulation, enabling the system to apply temperature corrections or calibration factors directly within the conversion equation. The processor may be configured to select between the polynomial conversion function and the lookup table based on various factors, such as the specific thermocouple type, temperature range, or detected system configuration. In some embodiments, the system may even dynamically switch between or combine both methods to optimize temperature measurement accuracy across different operating conditions.

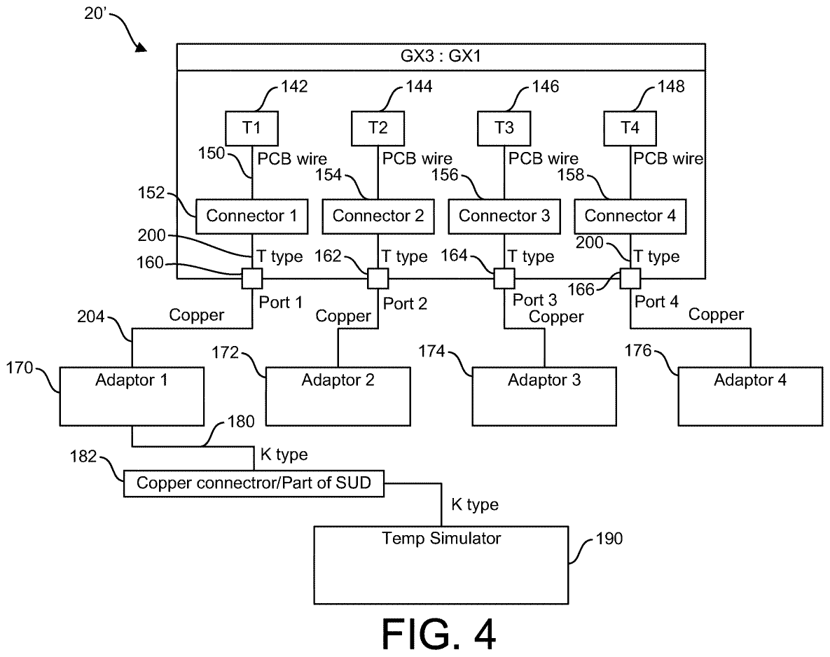

Having described the improved temperature measurement approach in FIG. 3, we now can examine the specific hardware configurations used to implement and test this universal method. FIG. 4 illustrates a schematic block diagram of a K-type Single Use Device (SUD) with adaptor thermal couple characterization configuration used to establish the correction factors for the system.

FIG. 4 illustrates a detailed schematic block diagram of a K-type probe (single use device (SUD)) with an adaptor thermal couple characterization configuration. This configuration represents one of the specific test setups used to determine the correction factors applied in the universal temperature measurement approach. The diagram shows the signal path from the temperature sensors inside the radiofrequency generator 20 to the probe tip where temperature is measured.

At the top of the diagram is a modified version of a GX1 radiofrequency generator 20′, which contains similar internal components for temperature measurement as previously described. Within the generator 20′ are four temperature sensors 142, 144, 146, and 148 (T1, T2, T3, and T4, respectively). The temperature sensors measure temperatures near the cold junction locations and provide the reference or cold junction voltage readings used in the voltage adjustment and correction technique described herein.

Each temperature sensor (142, 144, 146, 148) connects to a respective connector 152, 154, 156, and 158 (Connector1, Connector2, Connector3, and Connector4, respectively) via printed wire connections 150 disposed in a printed circuit board (PCB). The PCB wire connections 150 represent the physical conductive traces on the printed circuit board that carry the electrical signals between the temperature sensors and the connectors. The connections introduce thermal and electrical variations that the temperature measurement system addresses through the voltage adjustment and correction technique.

The connectors 152, 154, 156, and 158 (Connector1, Connector2, Connector3, and Connector4) are the internal termination points for the thermocouple circuits. Each connector contains T-type wiring 200, which means the internal wiring of the radiofrequency generator is configured for T-type thermocouples. This creates a material transition when K-type thermocouples are connected, necessitating specific corrections in the temperature measurement process.

The connectors 152, 154, 156, and 158 connect to external ports 160, 162, 164, and 166 (Port1, Port2, Port3, and Port4, respectively). These ports are the physical interfaces where external probes connect to the radiofrequency generator. The ports provide the mechanical and electrical connections for the probes, transferring the thermocouple signals to the internal measurement circuitry.

From the ports 160, 162, 164, and 166, copper connections 204 extend outward. Copper is used for these connections as a standard conductor that provides reliable electrical connections. The use of copper creates another material transition in the thermocouple circuit, contributing to measurement variations that the system corrects.

The copper connections 204 lead to adaptors 170, 172, 174, and 176 (shown as Adaptor1, Adaptor2, Adaptor3, and Adaptor4 respectively). The adaptors serve as interface components that enable K-type thermocouple probes to connect to the generator 20′ that is configured to include T-type internal wiring. The adaptors create material transitions in the thermocouple circuit, which affects the voltage-temperature relationship and are addressed in the temperature calculations of the voltage adjustment and correction technique.

By way of example, from Adaptor1 170, a K-type connection 180 can extend to a copper connector/part of SUD 182. The K-type connection 180 represents the thermocouple wiring specific to K-type thermocouples, while the copper connector/part of SUD 182 is the interface component on the Single Use Device probe. This configuration creates multiple material transitions that the temperature measurement system accounts for.

A temperature simulator 190 is also shown connected to the system. The temperature simulator 190 is a calibration device that generates precise, known temperatures or equivalent thermocouple voltages. It is used during the characterization process to establish the correction factors needed for accurate temperature measurement with different thermocouple types.

When the radiofrequency generator 20′ measures temperature using this configuration, the generator first obtains a voltage differential from the K-type thermocouple 182. The voltage signal when conveyed to the generator 20′ travels through multiple material transitions—from K-type thermocouple material in the thermocouple, through copper in the adaptor 170, to the T-type internal wiring 200 in the connector 152—before being digitized by the analog-to-digital converter in the radiofrequency generator.

The radiofrequency generator applies a first correction value to the digitized voltage reading to compensate for these material transitions. This linear correction transforms the voltage reading or value to accurately reflect the actual temperature at the probe tip, accounting for the voltage changes introduced by the mix of K-type, copper, and T-type materials in the electrical signal path.

Simultaneously, the system measures the temperature sensor 142 (T1) inside the radiofrequency generator 20′. Because this sensor is located on the PCB at a distance from the actual cold junction at connector 152, the system applies a second correction to compensate for this physical distance offset. This ensures that the cold junction reference temperature accurately represents the temperature at the junction where the thermocouple materials meet the standard copper conductors rather than at the location of the temperature sensor.

After applying both voltage corrections, the radiofrequency generator calculates or determines the adjusted temperature voltage and converts the voltage to a temperature value using a K-type-specific lookup table. The lookup table can include standardized conversion values appropriate for K-type thermocouples, ensuring accurate temperature measurement for this specific configuration.

The characterization process using this configuration involves setting the temperature simulator 190 to various known temperatures and recording the corresponding ADC readings and cold junction voltages from the system. By analyzing the relationships between the known temperatures and the measured values, the correction factors are derived and applied in the universal temperature measurement approach described in FIG. 3.

This configuration represents a challenge that may rise for temperature measurement, involving multiple material transitions. By applying corrections based on the physical characteristics of the system, accurate temperature measurement is achieved without the need for manufacturing calibration. The characterization process establishes correction factors that remain consistent across multiple devices, simplifying manufacturing while maintaining measurement accuracy.

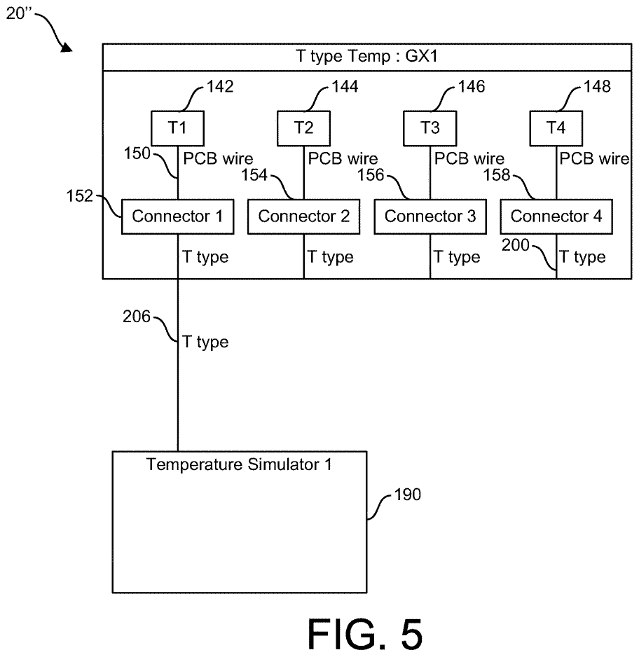

While FIG. 4 illustrates the configuration for K-type SUDs with adaptors, FIG. 5 presents a different configuration of the universal temperature measurement technique so as to accommodate T-type thermocouples. FIG. 5 shows a T-type thermal couple characterization configuration, which represents a direct connection arrangement for temperature measurement in the radiofrequency generator system.

FIG. 5 illustrates a schematic block diagram of a T-type thermal couple characterization configuration. This configuration represents a design where T-type thermocouples connect directly to the system without adaptors, creating a signal path with fewer material transitions.

At the top of the diagram is another configuration of the GX1 radiofrequency generator 20″, which contains similar internal components as shown previously. The temperature sensors 142, 144, 146, and 148 (labeled T1, T2, T3, and T4) are positioned on the printed circuit board inside the radiofrequency generator. These sensors measure temperatures that serve as reference points for cold junction compensation in the temperature calculation process.

Similar to FIG. 4, the temperature sensors connect to their respective connectors 152, 154, 156, and 158 (labeled Connector1, Connector2, Connector3, and Connector4) via PCB wire connections 150. The connectors contain T-type wiring 200, aligning with the internal architecture of the radiofrequency generator.

Instead of using an adaptor, the T-type thermocouple 206 connects directly to the connector through the port. This creates a more uniform signal path, as the thermocouple type corresponds to the internal wiring type of the radiofrequency generator. The direct T-type connection 206 reduces the material transitions that occur when using a K-type thermocouple with an adaptor. When the thermocouple materials match the internal wiring materials, there are fewer sources of measurement variation in this configuration. The system still applies corrections for the ADC reading and the cold junction voltage to achieve accurate measurements.

A temperature simulator1 190 is also shown connected to the system. Similar to FIG. 4, this simulator is used during the characterization process to establish the correction factors needed for accurate temperature measurement with T-type thermocouples.

In this T-type configuration, the temperature measurement system applies corrections that differ from those used in the K-type configuration. For the ADC reading, the system applies a linear correction specific to T-type thermocouples. This correction compensates for signal path characteristics within the radiofrequency generator, ensuring accurate digitization of the thermocouple voltage.

The cold junction voltage correction remains applicable in this more uniform configuration. The temperature sensors 142, 144, 146, and 148 are positioned at distances from their respective connectors 152, 154, 156, and 158, creating temperature offsets that benefit from compensation. The system applies channel-specific offset values to the measured cold junction voltages to account for these physical distances.

The universal temperature measurement approach accommodates both this T-type configuration and the K-type configuration shown in FIG. 4 by applying appropriate correction factors based on the thermocouple type. When a T-type thermocouple is detected, the system selects the T-type-specific corrections and lookup table, ensuring accurate temperature measurement.

By characterizing both configurations using their respective temperature simulators, the system establishes a comprehensive set of correction factors that can be applied based on the detected thermocouple type. This eliminates the need for individual device calibration during manufacturing while maintaining accurate temperature measurement across different thermocouple types and configurations.

The T-type configuration shown in FIG. 5 represents a configuration where the thermocouple type matches the internal wiring of the radiofrequency generator. The ability of the universal temperature measurement approach to accommodate both this matched configuration and the mixed-material configuration shown in FIG. 4 demonstrates the versatility of the system and its effectiveness with different thermocouple types without requiring manufacturing calibration.

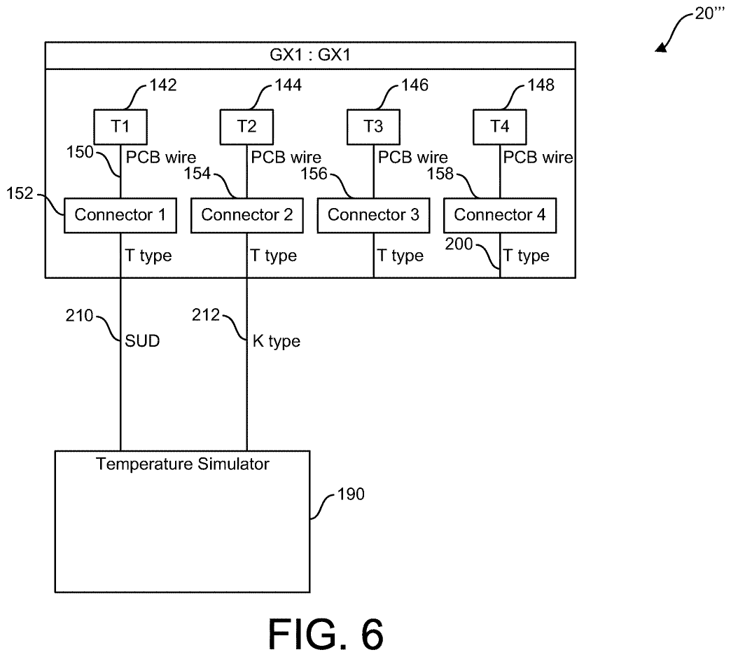

Moving from the T-type configuration, FIG. 6 illustrates a third configuration that further demonstrates the adaptability of the universal temperature measurement system. FIG. 6 shows a configuration with both a Single Use Device (SUD) and a direct K-type wiring thermal couple arrangement, representing an additional test setup used to characterize the system's performance with different thermocouple combinations. This configuration provides an alternative arrangement enabling for comparative analysis of different connection methods.

FIG. 6 shows another configuration of the GX1 radiofrequency generator 20″′, containing similar internal components seen in previous figures. The temperature sensors 142, 144, 146, and 148 (labeled T1, T2, T3, and T4) are positioned on the printed circuit board inside the radiofrequency generator, connecting to their respective connectors 152, 154, 156, and 158 via PCB wire connections 150. The connectors contain T-type wiring 200, consistent with the previous configurations.

In this configuration, two different connection methods are illustrated. From Connector1 152, a SUD connection 210 extends outward. This represents a Single Use Device probe connecting to the first port of the radiofrequency generator. From Connector2 154, a K-type connection 212 extends outward. This represents a direct K-type thermocouple connection to the second port of the radiofrequency generator.

A temperature simulator 190 is also shown connected to the system. The simulator provides standardized temperature references for both connection methods, enabling precise characterization of the system's response to each configuration.

The universal temperature measurement technique applies appropriate voltage corrections for each connection method. For the SUD connection 210, the system applies corrections that account for the material transitions and electrical characteristics specific to the SUD probe. For the direct K-type connection 212, the system applies corrections that account for the transition from K-type thermocouple material to T-type internal wiring within the radiofrequency generator.

This configuration demonstrates how the universal temperature measurement system can handle multiple thermocouple connections simultaneously, applying the appropriate corrections to each channel based on the detected thermocouple type and connection method. This capability enables the system to function in clinical settings where different probe types might be used for different procedures or on different channels.

The characterization process using this configuration helps establish correction factors that account for the variations between different K-type connection methods. By analyzing the relationships between known reference temperatures from the Temperature Simulator 190 and the measured values from each connection method, the system can derive correction factors that provide consistent performance across different connection arrangements.

The dual connection configuration shown in FIG. 6 complements the configurations shown in FIG. 4 and FIG. 5, providing a comprehensive characterization framework for the universal temperature measurement system. Together, these configurations enable the development of a unified approach to temperature measurement that accommodates different thermocouple types and connection methods without requiring individual device calibration during manufacturing.

This configuration illustrates how the universal temperature measurement system can be extended to handle additional thermocouple arrangements beyond the primary configurations shown in previous figures. By applying the same principles of characterized corrections for ADC readings and cold junction voltages, the system maintains accurate temperature measurement across a variety of probe types and connection methods.

Having examined the detailed configurations and characterization methods illustrated in FIGS. 1-6, the universal temperature measurement technique operates through a comprehensive approach that accommodates various thermocouple types and connection arrangements without requiring manufacturing calibration. The radiofrequency generator integrates several components and processes to achieve accurate temperature measurement across different configurations.

In operation, the radiofrequency generator receives a voltage signal from a thermocouple that is connected thereto. The voltage signal generated by the probe travels through the various material transitions in the connection path before being converted by the ADC in the generator to digital converter voltage signals. The generator then applies a first correction value to the digital converter voltage signals, using a linear formula with coefficients specific to the type of thermocouple to generate corrected converter voltage signals.

Simultaneously, the generator measures the temperature at the cold junction locations using temperature sensors positioned within the generator near the connector ports. These measurements are processed as cold junction voltage signals. Because the sensors are located at distances from the actual cold junctions, the generator applies a second correction value to compensate for the physical distance offsets to generate corrected cold junction voltage signals. The offset values are specific to each channel in the multi-channel design.

After applying both types of voltage corrections (e.g., digital converter voltage signals and to the cold junction voltage signalsc), the generator calculates an adjusted voltage and converts this voltage to a temperature value using a lookup table corresponding to the detected thermocouple type. This voltage adjustment and correction technique enables the generator to provide accurate temperature readings regardless of whether T-type or K-type thermocouples are connected thereto, and without requiring individual calibration of the generator during manufacturing.

The following embodiments further illustrate the scope and versatility of the universal temperature measurement system.

In some embodiment, the radiofrequency generator may include a user interface that displays the thermocouple type detected on each channel, allowing the user to verify that the system has correctly identified the connected probes.

In some embodiments, the system may include a calibration verification mode where a temperature simulator can be connected to verify the accuracy of the temperature measurement without requiring recalibration of the device.

In some embodiments, the system includes a diagnostic feature that monitors the stability of the cold junction voltage readings and alerts the user if significant fluctuations are detected that might affect measurement accuracy.

In some embodiments, the system may maintain historical temperature data in memory, enabling for analysis of temperature patterns during procedures and facilitating quality control measures.

In some embodiments, the system may apply different ADC correction formulas based on the temperature range being measured, using range-specific slope and intercept values to optimize accuracy across the full clinical temperature range.

In some embodiments, the system may include wireless connectivity to allow remote monitoring of temperature measurements and system diagnostics, facilitating technical support and quality assurance processes.

In some embodiments, the system may include a learning algorithm that refines the correction factors over time based on system performance, gradually optimizing the temperature measurement accuracy for the specific operating conditions of each device.

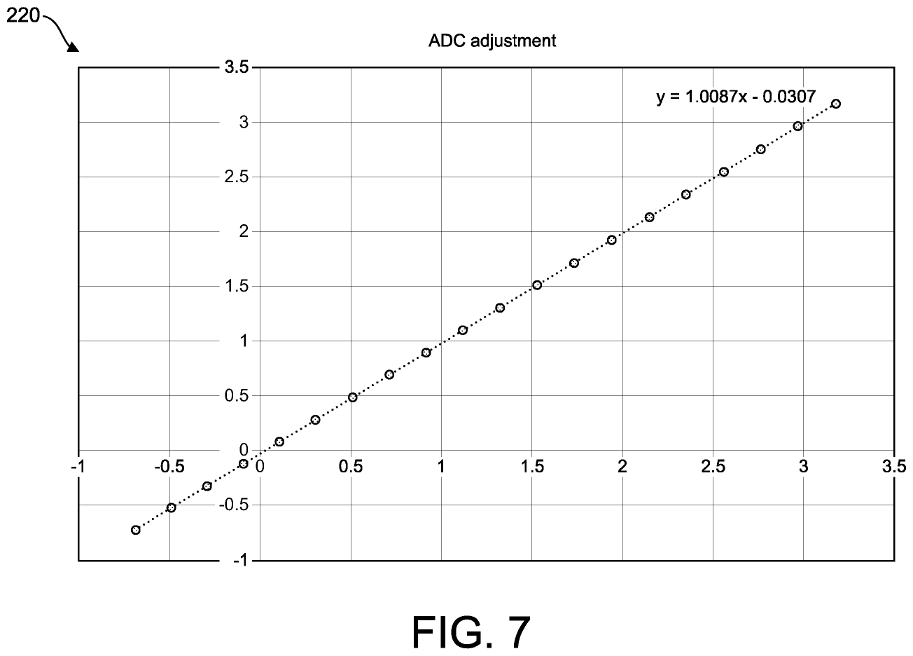

The previous figures illustrated the hardware configurations used for characterizing the temperature measurement system. FIG. 7 provides a graphical representation of the linear correction applied to ADC readings.

FIG. 7 illustrates a graph 220 showing the ADC adjustment relationship used in the first voltage correction of the voltage adjustment and correction technique. The graph 220 plots the relationship between the raw ADC readings (x-axis) and the corrected ADC values (y-axis), demonstrating the linear nature of the correction formula.

The graph 220 shows a linear relationship between the input and output values, represented by a straight line with numerous data points marked therealong. The data points represent actual measurement values collected during the characterization process, where known temperature inputs from a temperature simulator were compared with the corresponding ADC readings from the radiofrequency generator.

At the top of the graph 220, an example of the linear formula y=1.0087x−0.0307 is shown. This formula represents the mathematical relationship used for the first voltage correction applied to the ADC raw voltage values. In this formula, y represents the corrected ADC value, x represents the raw ADC reading, 1.0087 is the slope coefficient, and −0.0307 is the intercept value.

The slope coefficient of 1.0087 indicates that the correction slightly amplifies the raw ADC reading by a factor of just over 1. This adjustment compensates for minor attenuation in the signal path between the thermocouple and the analog-to-digital converter. The intercept value of −0.0307 represents a small offset correction that is applied to every reading, accounting for consistent bias in the measurement system.

The graph 220 spans a wide range of values, from approximately −1 to 3.5 on both axes, demonstrating that the linear correction applies consistently across the full measurement range used in clinical applications. The data points show close adherence to the linear formula, indicating that a simple linear correction provides accurate adjustment of the ADC readings.

This linear correction formula was derived through the characterization process illustrated in FIG. 4, FIG. 5, and FIG. 6. By comparing known input temperatures from the temperature simulator with the corresponding ADC readings across multiple devices, the system established this formula as a consistent correction factor that could be applied to all devices of the same design, eliminating the need for individual device calibration.

The linear correction illustrated in FIG. 7 represents just one component of the universal temperature measurement approach. When combined with the cold junction voltage correction and the appropriate thermocouple lookup table selection, this linear ADC correction enables accurate temperature measurement across different thermocouple types and connection configurations without requiring manufacturing calibration.

The consistency of the linear relationship across the measurement range demonstrates how a simple formula can correct the ADC readings. Rather than requiring complex, non-linear corrections or individual calibration curves for each device, this linear correction can be applied universally to all devices of similar design, simplifying the manufacturing process while maintaining measurement accuracy.

In the universal temperature measurement approach, this linear correction formula may vary depending on the thermocouple type being used. Different functions or different formulas with appropriate slope and intercept values can be applied based on the detected thermocouple type, ensuring accurate correction regardless of whether a T-type or K-type thermocouple is connected to the system.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A temperature measurement system, comprising:
a radiofrequency generator having at least one a power source, a controller operatively coupled to the at least one power source, a processor, a memory element, at least one analog-to-digital converter (ADC), and a plurality of connector ports, wherein each of the plurality of connector ports has at least one temperature sensor associated therewith;
a radiofrequency probe electrically coupled to a first one of the plurality of connector ports and having a thermocouple;
wherein the at least one ADC is configured to convert an analog voltage signal generated by the thermocouple into a digital converter voltage signal;
wherein the processor is configured to execute program instructions to:
determine a type of thermocouple used in the radiofrequency probe when coupled to the first one of the plurality of connector ports;
apply a first voltage correction value to the digital converter voltage signal based on a predetermined correction factor corresponding to the type of thermocouple to generate a corrected converter voltage signal;
receive temperature information from the at least one temperature sensor associated with the first one of the plurality of connector ports, wherein the temperature information includes a cold junction voltage signal;
apply a second voltage correction value to the cold junction voltage signal to compensate for a location offset of the at least one temperature sensor relative to the first one of the plurality of connector ports to generate a corrected cold junction voltage signal;
determine an adjusted voltage based on the corrected converter voltage signal and the corrected cold junction voltage signal; and
convert the adjusted voltage into a temperature value using a conversion technique.

2. The temperature measurement system of claim 1, wherein the first voltage correction value comprises a linear correction value with coefficient values specific to the type of thermocouple, wherein the linear correction value adjusts the digital converter voltage signal using a formula that includes a slope value multiplied by the digital converter voltage signal plus an intercept value.

3. The temperature measurement system of claim 2, wherein the second voltage correction value comprises a fixed temperature offset value for adjusting the cold junction voltage signal, wherein the fixed temperature offset value compensates for a physical distance between the temperature sensor and an actual cold junction location at a connector location of the first one of the plurality of connector ports.

4. The temperature measurement system of claim 1, wherein the first and second voltage correction values eliminate a need for individual radiofrequency probe calibration during manufacturing.

5. The temperature measurement system of claim 1, wherein the processor is further configured to automatically determine the type of thermocouple used in the radiofrequency probe; and select an appropriate voltage correction value based on the determined thermocouple type.

6. The temperature measurement system of claim 5, wherein determining the type of thermocouple comprises detecting one or more characteristics of the radiofrequency probe, wherein the characteristics comprise at least one of electrical resistance measurements, therapy type selections, pin configurations, voltage response patterns, or user interface inputs; and comparing the detected characteristics to predetermined reference values to identify the type of thermocouple.

7. The temperature measurement system of claim 1, wherein the processor is further configured to determine that the thermocouple is a K-type thermocouple; and select a K-type thermocouple lookup table for converting the adjusted voltage to the temperature value.

8. The temperature measurement system of claim 1, wherein the processor is further configured to determine that the thermocouple is a T-type thermocouple; and select a T-type thermocouple lookup table for converting the adjusted voltage to the temperature value.

9. The temperature measurement system of claim 1, wherein the radiofrequency probe comprises a single-use device (SUD) with a K-type thermocouple connected to the connector port via an adapter.

10. The temperature measurement system of claim 9, wherein the processor is further configured to apply a third voltage correction based on a combination of the SUD and an adapter when calculating the adjusted temperature voltage.

11. The temperature measurement system of claim 1, wherein the first voltage correction value compensates for electrical and thermal variations introduced by circuit paths between the temperature sensor and a connector location of the first one of the plurality of connector ports.

12. The temperature measurement system of claim 1, wherein the radiofrequency generator comprises multiple channels, wherein each channel corresponds to one of the plurality of connector ports, and wherein each channel has an independent temperature sensor offset value associated therewith.

13. The temperature measurement system of claim 1, wherein the processor is configured to apply different voltage correction values at different temperatures by selecting specific first and second voltage correction values from a set of predetermined voltage correction values characterized at multiple temperature conditions.

14. The temperature measurement system of claim 1, wherein the conversion technique comprises using a lookup table or a polynomial conversion function corresponding to the determined thermocouple type.

15. A method of measuring temperature in a tissue ablation system comprising a radiofrequency generator having an analog-to-digital converter (ADC) and a plurality of connector ports, wherein each of the plurality of connector ports has associated therewith a temperature sensor, and a radiofrequency probe having a thermocouple that is configured to be coupled to a first one of the plurality of connector ports, the method comprising:
determining a type of thermocouple used in the radiofrequency probe;
converting, by the ADC, an analog voltage signal from the thermocouple into a digital converter voltage signal;
applying a first voltage correction value to the digital converter voltage signal to generate a corrected converter voltage signal;
generating temperature information from the temperature sensor associated with the first one of the plurality of connector ports that is coupled to the radiofrequency probe, wherein the temperature information includes a cold junction voltage signal;
applying a second voltage correction value to the cold junction voltage signal to compensate for a location offset of the temperature sensor relative to the first one of the plurality of connector ports to generate a corrected cold junction voltage signal;
calculating an adjusted voltage based on the corrected converter voltage signal and the corrected cold junction voltage signal; and
converting the adjusted voltage to a temperature value.

16. The method of claim 15, wherein the first voltage correction comprise linear corrections with coefficient values specific to the thermocouple types, wherein the linear corrections adjust the digital converter voltage signals using formulas comprising slope values multiplied by the digital converter voltage signals plus intercept values.

17. The method of claim 15, wherein the second voltage correction value comprises a fixed temperature offset value for adjusting the cold junction voltage signal, wherein the fixed temperature offset value compensates for a physical distance between the temperature sensor and an actual cold junction location at a connector location of the first one of the plurality of connector ports.

18. The method of claim 15, further comprising automatically determining, by a processor of the generator, the type of thermocouple used in the radiofrequency probe; and selecting an appropriate voltage correction value based on the determined thermocouple type.

19. The method of claim 18, wherein determining the type of thermocouple comprises detecting, by the processor, one or more characteristics of the radiofrequency probe, wherein the one or more characteristics comprise at least one of electrical resistance measurements, therapy type selections, pin configurations, voltage response patterns, or user interface inputs; and identifying, by the processor, the type of thermocouple by comparing the detected characteristics to predetermined reference values stored in a memory of the radiofrequency generator.

20. The method of claim 15, wherein the conversion of the adjusted voltage to the temperature value is done using a look-up table or a polynomial conversion function corresponding to the determined thermocouple type.
